(19) United States
(12) Patent Application Publication
(10) Pub. No.: US 2010/0233740 A1
(43) Pub. Date: Sep. 16, 2010

(54) USE OF URINARY NGAL TO DISTINGUISH KIDNEY DISEASE AND PREDICT MORTALITY IN SUBJECTS WITH CIRRHOSIS

(76) Inventors: Jonathan BARASCH, New York, NY (US); Thomas L. NICKOLAS, New York, NY (US)

Correspondence Address: WilmerHale/Columbia University, 399 PARK AVENUE, NEW YORK, NY, 10022, US

(21) Appl. No.: 12/704,887

(22) Filed: Feb. 12, 2010

Related U.S. Application Data
(60) Provisional application No. 61/151,994, filed on Feb. 12, 2009.
(60) Provisional application No. 61/256,060, filed on Oct. 29, 2009.
(60) Provisional application No. 61/256,592, filed on Oct. 30, 2009.

(51) Int. Cl. G01N 33/53; G01N 33/566

(52) U.S. Cl. 435/7.92; 436/86; 436/501

(57) ABSTRACT

In one embodiment, the present invention is directed to methods for diagnosis of acute kidney injury (AKI) and hepatorenal syndrome (HRS) in cirrhosis patients, and to methods for distinguishing between AKI and/or HRS and/or other kidney diseases in cirrhosis subjects. In another embodiment, the present invention is directed to prognostic methods for predicting disease-specific mortality in cirrhosis patients. In some aspects, the diagnostic and prognostic methods of the invention are based on determining whether a bodily fluid sample, such as a urine sample, contains an amount of NGAL protein that exceeds or is less than a certain threshold level, or that falls within a certain range.

This application claims the benefit of the filing date of U.S. Provisional Patent Application No. 61/151,994, filed Feb. 12, 2009, U.S. Provisional Patent Application No. 61/256,060, filed Oct. 29, 2009, and U.S. Provisional Patent Application No. 61/256,592, filed Oct. 30, 2009, the contents of each of which are hereby incorporated by reference.

All patents, patent applications and publications cited herein are hereby incorporated by reference in their entirety.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Patients with liver disease, especially cirrhosis, often have changes in renal function. Some of the renal function change is due to a prerenal state whereby filtration decreases and recovery of the filtrate increases resulting in apparent renal failure. However, there are few histological changes and the apparent renal failure can be shown to reverse on improvement of blood flow. This referred to as hepatorenal syndrome (HRS). On the other hand, some patients with apparent renal failure do indeed have renal tubular damage. This is referred to as acute kidney injury (AKI). In patients with cirrhosis, AKI is an important predictor of mortality at the time of hospitalization. Currently, it is difficult to distinguish between types of renal disease in patients with liver disease such as cirrhosis. Serum creatinine (SCr) poorly distinguishes AKI from HRS, prerenal azotemia (PRA), or stable chronic kidney disease (CKD). The inability to distinguish between these conditions can lead to inappropriate therapy. Accordingly, there is a need for a biomarker to distinguish AKI from HRS and other kidney conditions in patients with cirrhosis.

### SUMMARY OF THE INVENTION

The present invention is based, in part, on certain discoveries which are described more fully in the Examples section of the present application. For example, the present invention is based, in part, on the discovery that levels of NGAL protein in the urine of cirrhosis patients with acute kidney injury (AKI) are much higher than the levels of NGAL in the urine of cirrhosis patients with other forms of kidney disease, such as hepatorenal syndrome (HRS), prenal azotemia (PRA) and chronic kidney disease (CKD), and on the discovery that the level of NGAL protein in the urine of cirrhosis patients is highly predictive or mortality. It is also a discovery of the present invention that NGAL levels are far superior to glomerular filtration rate (GFR) or serum creatinine levels for the purpose of distinguishing between AKI and other forms of kidney disease in cirrhosis patients and for the purposes of predicting mortality in cirrhosis patients.

In one embodiment the present invention provides a method for determining whether a subject with cirrhosis has AKI or a non-AKI kidney condition, comprising determining the concentration of NGAL protein in a urine sample from a subject with cirrhosis, wherein a concentration of NGAL in the urine sample that exceeds a threshold amount indicates that the subject has AKI, and wherein a concentration of NGAL in the urine sample that is less than the threshold amount indicates that the subject has a non-AKI kidney condition or has normal kidney function. In one such embodiment, the threshold amount of NGAL protein is between about 500 and about 1000 micrograms per gram creatinine. In one embodiment the non-AKI kidney condition is HRS, PRA or CKD.

In another embodiment the present invention provides a method for determining whether a subject with cirrhosis has HRS or a non-HRS kidney condition, comprising determining the concentration of NGAL protein in a urine sample from a subject with cirrhosis, wherein a concentration of NGAL in the urine sample that falls within a certain range indicates that the subject has HRS, and wherein a concentration of NGAL in the urine sample that is greater or less than the range indicates the subject has a non-HRS kidney condition or has normal kidney function. In one such embodiment, the range is from about 150 to about 500 micrograms per gram creatinine. In one embodiment a concentration of NGAL in the urine sample that is greater than the range indicates that the subject has AKI. In another embodiment a concentration of NGAL in the urine sample that is less than the range indicates the subject has PRA, CKD, or normal kidney function.

In another embodiment, the present invention provides methods for determining whether a subject with cirrhosis has AKI, HRS, or prerenal azotemia based on assessing uNGAL levels and also fractional excretion of urine sodium (FENa). In one embodiment, a stepwise analysis is performed. In a first step, subjects with PRA are differentiated from subjects with HRS or AKI based on measuring uNGAL levels, wherein subjects having a uNGAL level lower than a certain threshold amount have PRA, whereas subjects having a uNGAL level higher than the certain threshold amount have either AKI or HRS. In a second step, subjects with HRS are differentiated from subjects with AKI based on determining FENa values, wherein subjects having FENa values less than a certain threshold percentage have HRS, whereas subjects having FENa values greater than that threshold percentage have AKI. In one embodiment the threshold for the first step of differentiating patients with PRA is about 60 mg/mL—subjects having a concentration of NGAL in their urine of less than about 60 mg/mL having PRA. In one embodiment, the threshold percentage for step 2 is 0.5%, subjects having an FENa value of less than 0.5% having HRS as opposed to AKI.

In another embodiment, the present invention provides a method for predicting disease-specific mortality in a subject with cirrhosis, the method comprising determining the amount of NGAL protein in a urine sample from a subject with cirrhosis, wherein an amount of NGAL protein in the urine sample that exceeds a threshold amount indicates the subject has a high chance of disease-specific mortality, and an amount of NGAL protein in the urine sample that is less than the threshold amount indicates that the subject has a low chance of disease-specific mortality. In one such embodiment a subject with an amount of NGAL protein in the urine sample that exceeds the threshold amount has a 20- to 80-fold higher chance of disease-specific mortality than a subject having an amount of NGAL protein in the urine sample that is less than the threshold amount. In one such embodiment the threshold amount is between about 10 and about 180 micrograms per gram creatinine. In one embodiment the threshold amount is about 130 micrograms per gram creatinine. In one embodiment the urine sample is obtained during an acute episode of liver and/or kidney disease, and an amount of NGAL protein in the urine sample that exceeds the threshold amount indicates the subject has a high chance of disease-specific mortality during the acute episode, whereas an amount of NGAL protein in the urine sample that is less than the threshold amount indicates that the subject has a low chance of disease-specific mortality during the acute episode. In another embodiment the urine sample is obtained during a hospitalization for liver and/or kidney disease, and an amount of NGAL protein in the urine sample that exceeds the threshold amount indicates the subject has a high chance of disease-specific mortality during the hospitalization, whereas an amount of NGAL protein in the urine sample that is less than the threshold amount indicates that the subject has a low chance of disease-specific mortality during the hospitalization.

In some embodiments the methods of the invention comprise performing an immunoassay to detect NGAL protein.

In some embodiments the methods of the invention comprise adjusting the subject's treatment regimen based on whether the concentration of NGAL in the urine sample exceeds or is less than the chosen threshold amounts or ranges.

These and other embodiments of the invention are further described in the following sections of the application, including the Detailed Description, Examples, Claims, and Drawings.

### BRIEF DESCRIPTION OF THE FIGURES

FIG. 1 shows serum creatinine (mg/dL), eGFR (mL/min), and uNGAL levels (microgram/gram creatinine), stratified by category of renal function, for the patients in the study described in Example 2. * indicates p<0.05 as compared to AKI. † indicates <0.01 as compared to AKI. Patients with AKI had significantly greater serum creatinine compared to patients with PRA, CKD or normal kidney function. uNGAL was significantly elevated in AKI when compared to other categories of kidney function.

FIG. 2 shows the clinical outcomes of patients enrolled in the study described in the Examples. 16% of patients died, 18% were admitted to the intensive care unit, 14% required a nephrology consult, and 11% received renal replacement therapy.

FIG. 3 shows ROC curves for uNGAL and serum creatinine in predicting mortality and AKI in cirrhosis patients.

FIG. 4 shows serum creatinine (mg/dL), eGFR (mL/min), and uNGAL levels (microgram/gram creatinine), stratified by category of renal function, for the patients in the study described in Example 3. p values are as indicated. NS indicates no statistically significant difference.

### DETAILED DESCRIPTION

The present invention is based, in part, on certain discoveries which are described more fully in the Examples section of the present application. For example, the present invention is based, in part, on the discovery that levels of NGAL protein in the urine of cirrhosis patients with AKI are much higher than the levels of NGAL in the urine of cirrhosis patients with other forms of kidney disease, such as HRS, PRA and CKD, and on the discovery that the level of NGAL protein in the urine of cirrhosis patients is highly predictive or mortality. It is also a discovery of the present invention that measuring NGAL levels is far superior to measuring glomerular filtration rate (GFR) or serum creatinine levels for the purposes of distinguishing between AKI and other forms of kidney disease in cirrhosis patients and for the purposes of predicting mortality in cirrhosis patients. Thus, the present invention provides diagnostic and prognostic methods, and compositions and kits for use in diagnostic and prognostic methods.

### ABBREVIATIONS AND DEFINITIONS

The abbreviation “NGAL” refers to Neutrophil Gelatinase Associated Lipocalin. NGAL is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the term “NGAL”, as used herein, includes any NGAL protein, fragment, or mutant that is expressed in the kidney, and which can be detected in a bodily fluid such as urine. In some embodiments the NGAL protein is wild-type human NGAL.

The abbreviation “uNGAL” is an abbreviation for urinary NGAL and refers to NGAL in the urine.

The abbreviation “CKD” refers to chronic kidney disease.

The abbreviation “AKI” refers to acute kidney injury.

The abbreviation “HRS” refers to hepatorenal syndrome.

The abbreviation “PRA” refers to prerenal azotemia.

The abbreviation “SCr” refers to serum creatinine.

The abbreviation “FENa” refers to fractional excretion of sodium.

The abbreviation “ESRD” refers to end-stage renal disease.

The abbreviation “GFR” refers to glomerular filtration rate.

The abbreviation “eGFR” refers to estimated glomerular filtration rate.

The abbreviation “MELD” refers to the model for end-stage liver disease. The MELD scoring system for assessing prognosis in liver disease is well known in the art. See, for example, Freeman et al., “Improving liver allocation: MELD and PELD; American Journal of Transplantation, 4 Suppl 9: 114-31, the contents of which are hereby incorporated by reference.

The abbreviation “MDRD” refers to modification of diet in renal disease The MDRD equation for calculating GFR and/or eGFR is well known in the art. See, for example, Levey et al., (1999), “A more accurate method to estimate glomerular filtration rate from serum creatinine: a new prediction equation. Modification of Diet in Renal Disease Study Group.” Ann. Intern. Med. 1999; 130: 461-470, and Levey et al., (2000), “A simplified equation to predict glomerular filtration rate from serum creatinine” J. Am. Soc. Nephrol. 2000; (11):155A, the contents of which are hereby incorporated by reference.

The abbreviation “ROC” refers to receiver operating characteristic. ROC curves are widely used in the art for assessing diagnostic and prognostic tests. See, for example, Zweig & Campbell, (1993), “Receiver-operating characteristic (ROC) plots: a fundamental evaluation tool in clinical medicine”. Clinical chemistry 39 (8): 561-577; and Zou et al., (2007). “Receiver-operating characteristic analysis for evaluating diagnostic tests and predictive models.” Circulation, 6; 115 (5): 654-7; and Lasko et al., (2005), “The use of receiver operating characteristic curves in biomedical informatics.” Journal of Biomedical Informatics, 38 (5):404-415, the contents of each which are hereby incorporated by reference.

The abbreviation “AUC” refers to area under the curve.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Unless stated otherwise, the term “mortality” as used herein refers to disease-specific mortality, such as, for example, mortality resulting from a patients cirrhosis and/or kidney condition as opposed to some other cause.

### DESCRIPTION

In one aspect of the invention, levels of NGAL protein in a bodily fluid, such as urine, that exceed a certain threshold amount can be used to diagnose AKI in cirrhosis patients and to distinguish AKI from other forms of kidney disease. It is a discovery of the invention that, in the two populations of cirrhosis patients tested (see Examples 1 and 3), patients with AKI had a mean uNGAL concentration of 1069 or 1173 μg/g creatinine, patients with HRS had a mean uNGAL concentration of 178 or 318 μg/g creatinine, patients with PRA had a mean uNGAL concentration of 26 or 67 μg/g creatinine, patients with CKD had a mean uNGAL concentration of 33 or 111 μg/g creatinine, and patients with normal kidney function had a mean uNGAL concentration of 27 or 58 μg/g creatinine. Thus, cirrhosis patients with AKI have elevated uNGAL concentrations, having much higher uNGAL concentrations than cirrhosis patients with HRS, PRA, CKD, or with normal kidney function. Cirrhosis patients with HRS also have elevated uNGAL concentrations, having uNGAL concentrations that are higher than those of cirrhosis patients with PRA, CKD, or with normal kidney function.

Accordingly, in one embodiment the present invention provides methods for determining whether a subject with cirrhosis has AKI, the methods comprising measuring the amount of NGAL protein in a bodily fluid, such as urine, from the subject, wherein an amount of NGAL protein that exceeds a threshold level, such as a threshold level of about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, or about 750 μg/g creatinine, or about 800 μg/g creatinine, or about 850 μg/g creatinine, or about 900 μg/g creatinine, or about 950 μg/g creatinine, or about 1000 μg/g creatinine, indicates that the subject has AKI. Conversely, an amount of NGAL protein that is less than the threshold level can indicate that the subject has a non-AKI kidney disease or condition or normal kidney function.

In another embodiment, the present invention provides methods for distinguishing between AKI and non-AKI kidney conditions in a subject with cirrhosis. Such methods comprise measuring the amount of NGAL protein in a bodily fluid, such as urine, from the subject, wherein an amount of NGAL protein that exceeds a threshold level, such as a threshold level of about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, or about 750 μg/g creatinine, or about 800 μg/g creatinine, or about 850 μg/g creatinine, or about 900 μg/g creatinine, or about 950 μg/g creatinine, or about 1000 μg/g creatinine, indicates that the subject has AKI. Conversely, an amount of NGAL protein that is less than the threshold level can indicate that the subject has a non-AKI kidney disease or condition or normal kidney function.

In one embodiment the above methods can be used for the early detection of AKI, for example, before the onset of symptoms of AKI, e.g. in an cirrhosis patient who is at risk for developing AKI. Accordingly, in one aspect, the above methods be used to diagnose AKI in a subject who is not exhibiting signs of AKI.

In another embodiment, the present invention provides a method for monitoring the progression of AKI in a subject with cirrhosis, the method comprising measuring the amount of NGAL protein in a first bodily fluid sample taken from the subject and a second bodily fluid sample that is taken from the subject at a later period in time, wherein an amount of NGAL protein in the second sample that exceeds the amount of NGAL protein in the first sample, indicates that the AKI is worsening, and an amount of NGAL protein in the second sample that is less than the amount of NGAL protein in the first sample, indicates that the AKI is improving. In one embodiment, the first sample can be taken before the initiation of therapy for kidney disease and/or for liver disease, and the second sample can be taken after the initiation of such therapy. In another embodiment, both samples can be taken after the initiation of therapy. Thus, such methods can be used to monitor the effect of therapy, such as therapy for kidney disease and/or liver disease, on the progression of AKI in a subject.

In yet another embodiment, the present invention provides a solution to the problem of determining whether a subject is a candidate for treatment of AKI, the method comprising measuring the amount of NGAL protein in a bodily fluid, such as urine, from the subject, wherein an amount of NGAL protein that exceeds a threshold level, such as a threshold level of about 400 μg/g creatinine, or about 450 μg/g creatinine, or about 500 μg/g creatinine, or about 550 μg/g creatinine, or about 600 μg/g creatinine, or about 650 μg/g creatinine, or about 700 μg/g creatinine, or about 750 μg/g creatinine, or about 800 μg/g creatinine, or about 850 μg/g creatinine, or about 900 μg/g creatinine, or about 950 μg/g creatinine, or about 1000 μg/g creatinine, indicates that the subject has is a candidate for treatment of AKI. Conversely an amount of NGAL protein that is less than the threshold level can indicate that the subject is not a candidate for treatment of AKI. In other embodiments, such methods also comprise subsequently treating the subject.

Another aspect of the invention provides a method of monitoring the effectiveness of a treatment for AKI in subject with cirrhosis, the method comprising the steps of: i) obtaining a baseline sample of a body fluid, such as urine, from the subject, ii) determining the level of NGAL in the baseline sample; iii) providing at least one treatment for the AKI; iv) obtaining at least one post-treatment sample of the body fluid from the subject; v) determining the level of NGAL in the post-treatment sample; and vi) evaluating the effectiveness of the treatment, based on comparing the level of NGAL in the post-treatment sample to the level of NGAL in the baseline sample.

It should be noted that in all of the embodiments above that deal with making an assessment relating to AKI based on detecting a level of NGAL in the urine that exceeds the recited threshold amounts, ranges of uNGAL amounts can be used in the place of threshold values. For example, in the above embodiments, threshold ranges can be substituted with ranges, such as an amount of uNGAL that falls within the range of about 400-1500 μg/g creatinine, or about 450-1500 μg/g creatinine, or about 500-1500 μg/g creatinine, or about 550-1500 μg/g creatinine, or about 600-1500 μg/g creatinine, or about 650-1500 μg/g creatinine, or about 700-1500 μg/g creatinine, or about 750-1500 μg/g creatinine, or about 800-1500 μg/g creatinine, or about 850-1500 μg/g creatinine, or about 900-1500 μg/g creatinine, or about 950-1500 μg/g creatinine, or about 1000-1500 μg/g creatinine. Also, the upper end of each of the preceding ranges can be adjusted, for example to about 1200 μg/g creatinine, or about 1300 μg/g creatinine, or about 1400 μg/g creatinine, or about 1600 μg/g creatinine, or about 1700 μg/g creatinine, or about 1800 μg/g creatinine, or about 1900 μg/g creatinine, or about 2000 μg/g creatinine, or about 2500 μg/g creatinine.

In another embodiment, the present invention provides methods for determining whether a subject with cirrhosis has AKI, HRS, or prerenal azotemia based on assessing uNGAL levels and also determining fractional excretion of urine sodium (FENa). In one embodiment, a stepwise analysis is performed. In a first step, subjects with PRA are differentiated from subjects with HRS or AKI based on measuring uNGAL levels, wherein subjects having a uNGAL level lower than a certain threshold amount have PRA, whereas subjects having a uNGAL level higher than the certain threshold amount have either AKI or HRS. In a second step, subjects with HRS are differentiated from subjects with AKI based on determining FENa values, wherein subjects having FENa values less than a certain threshold percentage have HRS, whereas subjects having FENa values greater than that threshold percentage have AKI.

In one embodiment, the uNGAL threshold for performing the first step of the above stepwise analysis is about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, or about 100 mg/mL uNGAL. In one embodiment the threshold is between about 40 mg/mL to about 80 mg/mL uNGAL. In one embodiment the threshold is between about 50 mg/mL to about 70 mg/mL. In a preferred embodiment, the threshold is about 60 mg/mL NGAL. Subjects having a concentration of NGAL in their urine of less than this threshold are likely to have PRA.

In one embodiment, the threshold FENa percentage for performing the second step of the above stepwise analysis, is about 0.1%, 0.2%, 0.3%, 0.4%, 0.5%, 0.6%, 0.7%, 0.8%, 0.9%, or 1.0%. In a preferred embodiment, the threshold is about 0.5%. Subjects having an FENa value of less than this threshold percentage are likely to have HRS as opposed to AKI.

Fractional excretion of urine sodium or “FENa” is a measure of the percentage of sodium excreted in the urine versus the sodium reabsorbed by the kidney. It is measured in terms of plasma and urine sodium, rather than by the interpretation of urinary sodium concentration alone, as urinary sodium concentrations can vary with water resorption. FENa calculations are performed routinely by those skilled in the art. See, for example, Steiner R (1984) “Interpreting the fractional excretion of sodium,” Am J Med 77 (4): 699-702, the contents of which are hereby incorporated by reference. In order to calculate FENa values, the concentration of sodium (Na) and creatinine (Cr) in each of the plasma (P) and the urine (U) must be determined. To accurately interpret FENa, subjects should not have recently received diuretics. FENa can be calculated based on these four measurements using the equation:

$${FE}_{Na} = \frac{{U}_{Na} * {P}_{Cr}}{{P}_{Na} * {U}_{Cr}} \times 100$$

In one aspect of the invention, levels of NGAL protein in the urine that exceed a certain threshold amount can be used to diagnose HRS in cirrhosis patients and to distinguish HRS from other forms of kidney disease. As described above, it is a discovery of the invention that, in the two populations of cirrhosis patients tested (see Examples 1 and 3) patients with AKI had a mean uNGAL concentration of 1069 or 1173 μg/g creatinine, patients with HRS had a mean uNGAL concentration of 178 or 318 μg/g creatinine, patients with PRA had a mean uNGAL concentration of 26 or 67 μg/g creatinine, patients with CKD had a mean uNGAL concentration of 33 or 111 μg/g creatinine, and patients with normal kidney function had a mean uNGAL concentration of 27 or 58 μg/g creatinine. Thus, cirrhosis patients with HRS have elevated uNGAL concentrations as compared to cirrhosis patients with PRA, CKD, or with normal kidney function, and have lower uNGAL concentrations as compared to cirrhosis patients with AKI.

Accordingly, the present invention provides methods for determining whether a subject with cirrhosis has HRS, the methods comprising measuring the amount of NGAL protein in a bodily fluid, such as urine, from the subject, wherein an amount of NGAL protein that exceeds a threshold level, such as a threshold level of about 100 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine indicates that the subject has HRS. Conversely, an amount of NGAL protein that is less than the threshold level can indicate that the subject has a non-HRS kidney disease or condition such as PRA or CKD.

In another embodiment, the present invention provides methods for distinguishing between HRS and non-HRS kidney conditions in a subject with cirrhosis. Such methods comprise measuring the amount of NGAL protein in a bodily fluid, such as urine, from the subject, wherein an amount of NGAL protein that exceeds a threshold level, such as a threshold level of about 100 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine indicates that the subject has HRS. Conversely, an amount of NGAL protein that is less than the threshold level can indicate that the subject has a non-HRS kidney disease or condition such as PRA or CKD.

In one embodiment the above methods can be used for the early detection of HRS, for example, before the onset of symptoms of HRS, e.g. in an cirrhosis patient who is at risk for developing HRS. Accordingly, in one aspect, the above methods be used to diagnose HRS in a subject who is not exhibiting signs of kidney disease.

In another embodiment, the present invention provides a method for monitoring the progression of HRS in a subject with cirrhosis, the method comprising measuring the amount of NGAL protein in a first bodily fluid sample taken from the subject and a second bodily fluid sample that is taken from the subject at a later period in time, wherein an amount of NGAL protein in the second sample that exceeds the amount of NGAL protein in the first sample, indicates that the HRS is worsening, and an amount of NGAL protein in the second sample that is less than the amount of NGAL protein in the first sample, indicates that the HRS is improving. In one embodiment, the first sample can be taken before the initiation of therapy for kidney disease and/or for liver disease, and the second sample can be taken after the initiation of such therapy. In another embodiment, both samples can be taken after the initiation of therapy. Thus, such methods can be used to monitor the effect of therapy, such as therapy for kidney disease and/or liver disease, on the progression of HRS in a subject.

In yet another embodiment, the present invention provides a solution to the problem of determining whether a subject is a candidate for treatment of HRS, the method comprising measuring the amount of NGAL protein in a bodily fluid, such as urine, from the subject, wherein an amount of NGAL protein that exceeds a threshold level, such as a threshold level of about 100 μg/g creatinine, or about 150 μg/g creatinine, or about 200 μg/g creatinine, or about 250 μg/g creatinine, or about 300 μg/g creatinine, or about 350 μg/g creatinine, or about 400 μg/g creatinine indicates that the subject is a candidate for treatment of HRS. Conversely an amount of NGAL protein that is less than the threshold level can indicate that the is not a candidate for treatment of HRS. In other embodiments, such methods also comprise subsequently treating the subject.

Another aspect of the invention provides a method of monitoring the effectiveness of a treatment for HRS in subject with cirrhosis, the method comprising the steps of: i) obtaining a baseline sample of a body fluid, such as urine, from the subject, ii) determining the level of NGAL in the baseline sample; iii) providing at least one treatment for the HRS; iv) obtaining at least one post-treatment sample of the body fluid from the subject; v) determining the level of NGAL in the post-treatment sample; and vi) evaluating the effectiveness of the treatment, based on comparing the level of NGAL in the post-treatment sample to the level of NGAL in the baseline sample.

It should be noted that in all of the embodiments above that deal with making an assessment relating to HRS based on detecting a level of NGAL in the urine that exceeds the recited threshold amounts, ranges of uNGAL amounts can be used in the place of threshold values. For example, in the above embodiments, threshold ranges can be substituted with ranges, such as an amount of uNGAL that falls within the range of about 100-400 μg/g creatinine, or about 150-400 μg/g creatinine, or about 200-400 μg/g creatinine, or about 250-400 μg/g creatinine, or about 300-400 μg/g creatinine, or about 350-1500 μg/g creatinine. Also, the upper end of each of the preceding ranges can be adjusted, for example to about 500 μg/g creatinine, or about 600 μg/g creatinine, or about 700 μg/g creatinine, or about 800 μg/g. The upper end of such a range should be chosen so that most patients having a uNGAL measurement within that range have HRS and not AKI.

It is a discovery of the present invention that a urinary NGAL measurement of 130 micrograms/gram creatinine, as measured on hospital admission, is highly predictive of inpatient mortality during that hospital admission—the sensitivity of inpatient mortality prediction being around 57% and the specificity of inpatient mortality prediction being around 98%. It is also a discovery of the invention, that the odds of death during the same hospital admission are around 48-fold higher for patients with a uNGAL measurement of greater than 130 micrograms/gram creatinine than for patients with a uNGAL measure of less than 130 micrograms/gram creatinine, as measured on hospital admission. It is also a discovery of the invention that for each 10 microgram/gram creatine rise in uNGAL, the odds of mortality increases by 17%. Thus, in one aspect, the present invention provides prognostic methods for predicting mortality in a subject with cirrhosis.

In some embodiments, a cirrhosis patient's uNGAL is measured when presenting with an acute episode liver and/or kidney related disease, and a uNGAL measurement that exceeds a certain threshold level indicates that the patient has a high chance of disease-specific mortality during that acute episode.

In some embodiments, a cirrhosis patient's uNGAL is measured during hospitalization for an acute episode liver and/or kidney related disease, and a uNGAL measurement that exceeds a certain threshold level indicates that the patient has a high chance of mortality during that hospitalization (i.e. a high chance of inpatient mortality).

In some embodiments, a cirrhosis patient's uNGAL is measured when presenting with an acute episode liver and/or kidney related disease, and a uNGAL measurement that exceeds a certain threshold level indicates that the patient has a high chance of disease-specific mortality within a certain period of time, for example within a month, two months, three months, six months, nine months, twelve months, fifteen months, eighteen months, two years, three years, four years, or five years.

In various embodiments, the present invention provides methods for predicting disease-specific mortality of a cirrhosis patient, based on (a) determining a level of NGAL in a bodily fluid sample, such as a urine sample, from the patient; and (b) comparing the level of NGAL against a predetermined NGAL cut-off level, wherein an NGAL level above the cut-off level indicates that the subject has a high chance of mortality, and wherein a NGAL level below the cut-off level indicates that the patient has a low chance of mortality/death.

In one embodiment, the cut-off level for mortality prediction is a uNGAL measurement of about 130 micrograms/gram creatinine.

In other embodiments, the uNGAL cut-off value for predicting mortality can be varied. A uNGAL cut-off value that gives a desired statistical outcome, for example a desired or acceptable sensitivity and specificity for mortality prediction, can be used. In preferred embodiments, the uNGAL cut-off is in the range of 100-180 micrograms uNGAL/gram creatinine. For example, the uNGAL cut-off can be about 100 micrograms uNGAL/gram creatinine, or about 105 micrograms uNGAL/gram creatinine, or about 110 micrograms uNGAL/gram creatinine, or about 115 micrograms uNGAL/gram creatinine, or about 120 micrograms uNGAL/gram creatinine, or about 125 micrograms uNGAL/gram creatinine, or about 130 micrograms uNGAL/gram creatinine, or about 135 micrograms uNGAL/gram creatinine, or about 140 micrograms uNGAL/gram creatinine, or about 145 micrograms uNGAL/gram creatinine, or about 150 micrograms uNGAL/gram creatinine, or about 155 micrograms uNGAL/gram creatinine, or about 160 micrograms uNGAL/gram creatinine, or about 165 micrograms uNGAL/gram creatinine, or about 170 micrograms uNGAL/gram creatinine, or about 175 micrograms uNGAL/gram creatinine, or about 180 micrograms uNGAL/gram creatinine.

In other embodiment the uNGAL cut-off level for mortality prediction can be, for example, about 50 micrograms uNGAL/gram creatinine, or about 60 micrograms uNGAL/gram creatinine, or about 70 micrograms uNGAL/gram creatinine, or about 80 micrograms uNGAL/gram creatinine, or about 90 micrograms uNGAL/gram creatinine, or about 190 micrograms uNGAL/gram creatinine, or about 200 micrograms uNGAL/gram creatinine, or about 225 micrograms uNGAL/gram creatinine, or about 250 micrograms uNGAL/gram creatinine, or about 275 micrograms uNGAL/gram creatinine, or about 300 micrograms uNGAL/gram creatinine, or about 350 micrograms uNGAL/gram creatinine, or about 400 micrograms uNGAL/gram creatinine, or about 450 micrograms uNGAL/gram creatinine, or about 500 micrograms uNGAL/gram creatinine.

A “high chance or mortality” can mean that a subject has a chance of mortality that is about 30-70 fold higher than the chance of mortality (e.g. inpatient mortality) of a another subject, such as a cirrhosis patient with no kidney disease, or a cirrhosis patient with non-AKI kidney disease, or a cirrhosis patient that has a uNGAL measurement below a certain threshold level. For example, a subject with a high chance of disease-specific mortality can have a chance or mortality that is about 20, 25, 30, 35, 40, 45, 50, 55, 60, 65, 75, or 80-fold higher than the chance of disease-specific mortality of a subject with no kidney disease, or a subject with non-AKI kidney disease, or a subject that has a uNGAL measurement below a certain threshold level.

All of the diagnostic and prognostic methods described herein can be combined in various ways. Furthermore, the following description applies to all of the diagnostic and prognostic methods described herein.

All of the diagnostic and prognostic methods of the invention, such as those described above, can comprise one or more additional steps. The diagnostic and/or prognostic methods of the invention may comprise one or more steps for obtaining the bodily fluid sample from the subject, for example using the methods described herein. The diagnostic and/or prognostic methods of the invention may comprise one or more steps for treating the bodily fluid sample from the subject, for example using the methods described herein. The diagnostic and/or prognostic methods of the invention may comprise one or more steps for detecting and/or measuring NGAL levels in the bodily fluid sample, for example using the methods described herein. The diagnostic and/or prognostic methods of the invention may comprise one or more steps for treating the subject or altering the subject's treatment based on the level of NGAL detected and/or whether the measured NGAL level is greater or less than the chosen cut-off level. For example, if the subject's NGAL level exceeds a cut-off level for distinguishing AKI from other forms of kidney disease, the subject may be treated for AKI. Similarly, depending on whether a subject's NGAL level exceeds or is less than a cut-off level chosen for mortality prediction, the subject may be treated differently, e.g. more or less aggressively, or may be offered palliative and/or hospice care, and/or may be offered psychotherapy or counseling.

According to the methods of the invention, such as the diagnostic and prognostic methods described above, the bodily fluid can be any sample in which NGAL can be detected, including, but not limited to, blood, serum, or urine. In some embodiments the bodily fluid is urine.

Also according to the methods of the invention, the subject can be any animal that is susceptible to cirrhosis. In some embodiments the subjects are rodents, such as mice. In some embodiments, the subjects are cows, pigs, sheep, goats, cats, horses, dogs, and/or any other species of animal used as livestock or kept as pets. In some embodiments the subjects are human subjects. In preferred embodiments, the subjects have already been diagnosed with cirrhosis. The subjects can have already been diagnosed with AKI or HRS or may not have been diagnosed with AKI or HRS.

In certain embodiments, the NGAL protein detected and/or measured in the methods of the present invention has an amino acid sequence as defined by one of the following GenBank accession numbers, NP_{—}005555 (human NGAL), CAA67574 (human NGAL), P80188 (human NGAL), AAB26529 (human NGAL), P11672 (mouse NGAL), P30152 (rat NGAL), AAI132070 (mouse NGAL), AAI132072 (mouse NGAL), AAH33089 (human NGAL), and CAA58127 (human NGAL), or is a homolog, variant, derivative, fragment, or mutant thereof, and/or has at least 80% sequence identity, e.g., 85%, 90%, 95%, 98% or 99% sequence identity, with one of the above sequences.

In certain embodiments of the invention, it can be desirable to use a positive control for the detection of NGAL. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

As described above, in certain embodiments, the present invention provides methods for determining whether a subject with cirrhosis has AKI or HRS, and methods for predicting the mortality of a subject with cirrhosis, such methods comprising measuring the amount of NGAL protein in a bodily fluid, such as urine, from the subject, wherein an amount of NGAL protein that exceeds a threshold level indicates that the subject has AKI or HRS or provides an indication about the subject's mortality. In addition to the threshold levels specified herein, a threshold level can also be selected by reviewing the data provided in the Examples section of this application, so that the threshold level is sufficiently high that it is more likely than not that a subject having that level of NGAL will have AKI as opposed to any other form of kidney disease, or will have HRS as opposed to any other form of kidney disease, and/or so that the threshold level is sufficiently high that it indicates that, more likely than not, that subject will be in a poor prognostic category and/or has a high chance or mortality/death.

It should also be noted that, although the amounts of NGAL described herein are generally referred to in terms of the amount by mass of NGAL relative to the amount by mass of creatinine, e.g. NGAL μg/g creatinine, NGAL can also be measured and/or represented in other units, including, but not limited to measurements of the amount of NGAL by mass (e.g. in nanograms or micrograms), or measurements of the amount of NGAL by concentration (e.g. in ng/mL), or any other units, and it should be understood that amounts of NGAL measured and/or represented in other units can be equivalent to the amounts and ranges described herein in terms of μg/g creatinine. The present invention is not limited to methods that comprise measuring NGAL and also measuring creatinine and/or to methods that comprise calculating the amount of NGAL in a sample of bodily fluid in terms of the number of micrograms per gram of creatinine. For example, an amount of NGAL that is represented herein as 100 μg/g creatinine, can also be represented in terms of, and encompasses, alternative measurements/units that correspond to the same amount of NGAL, e.g. the same amount of NGAL expressed in terms of mass (e.g. ng), or in terms of concentration (e.g. ng/mL) or in any other units. One of skill in the art can readily make the necessary conversions between units.

Furthermore, it should be noted that threshold levels or ranges of NGAL other than those specifically described herein may be used in accordance with the invention. It is a discovery of the invention that NGAL levels are higher in the urine of subjects with AKI as compared to subjects without HRS, and are higher in subjects with HRS as compared to subjects with other kidney conditions. The mean levels of uNGAL in such groups (e.g. control, HRS, and AKI groups) may vary in different groups of subjects or depending on the methodology used to measure NGAL levels. Accordingly, the present invention provides for the general concept of using uNGAL levels to diagnose AKI or HRS, and to distinguish AKI from HRS, and to predict mortality, and not only methods that rely on the specific thresholds and ranges provided herein.

In certain embodiments, other biomarkers can be assessed in addition to NGAL in order to determine whether a subject has AKI or HRS or to predict mortality. For example, the present invention provides that, in addition to having a high level of urinary NGAL, cirrhosis patients can also have one or more of: (i) proteinuria, (ii) a high serum creatinine level, (iii) a low glomerular filtration rate, and/or (iv) a high MELD score. Thus, in some embodiments the present invention provides methods comprising measuring the amount of NGAL in the urine of the subject and also (i) determining whether the subject has proteinuria, (ii) determining the subject's serum creatinine level, (iii) determining the subject's glomerular filtration rate, and/or calculating the subject's MELD score. Such additional biomarkers can be used in conjunction with the methods of diagnosing AKI and HRS, and the methods of predicting mortality, provided herein.

For example, in one embodiment, the combination of a high urinary NGAL measurement, i.e. a level an amount of NGAL in the urine that exceeds one of the threshold amounts described herein, together with a determination that the subject has proteinuria indicates that the subject can have AKI. The presence of any amount of protein (typically albumin) in the urine, in combination with a high urinary NGAL level, can be indicative of AKI. For example, the amount of protein (typically albumin) in the urine can be greater than about 0.1 mg/dl, or greater than about 0.2 mg/dl, or greater than about 0.3 mg/dl, or greater than about 0.4 mg/dl, or greater than about 0.5 mg/dl, or greater than about 0.6 mg/dl, or greater than about 0.7 mg/dl, or greater than about 0.8 mg/dl, or greater than about 0.9 mg/dl, or greater than about 1.0 mg/dl, or greater than about 1.1 mg/dl, or greater than about 1.2 mg/dl, or greater than about 1.3 mg/dl. Methods of determining whether a subject has proteinuria are well known in the art and are routinely performed by medical professionals, for example using standard laboratory tests or using urine dipstick methods. Most proteinuria tests are based on the detection of albumin in the urine, and/or by determining the level of albumin in the urea as a function of the urine creatinine level, e.g. determining the urine albumin-to-creatinine ratio (UACR). Any proteinuria test can be used in conjunction with the methods of the present invention.

In another embodiment, the combination of a high urinary NGAL measurement, i.e. a level an amount of NGAL in the urine that exceeds one of the threshold amounts indicated above, together with a serum creatinine level that is more than about 1 mg/dl, or more than about 1.5 mg/dl, or more than about 2.0 mg/dl or more than about or more than about 2.5 mg/dl, or more than about 3 mg/dl, or more than about 4 mg/dl indicates that the subject may have AKI. Methods of determining the serum creatinine level of a subject are well known in the art and are routinely performed by medical professionals. Any method for determining a subject's serum creatinine level can be used in conjunction with the methods of the present invention. A review of some methods for determining serum creatinine levels is provided in: Clin. Biochem. Rev. (2006); 27 (4): p 173-184; “Measurement of Serum Creatinine—Current Status and Future Goals” by Peake et al., the contents of which are hereby incorporated by reference.

According to the methods of the invention, samples of a bodily fluid can be obtained and/or tested using any means. For example, methods for collecting, handling and processing urine, blood, serum and plasma, and other body fluids, are well known in the art and can be used in the practice of the present invention. In some embodiments, two or more consecutive or subsequent samples of a body fluid can be taken. Depending upon the circumstances, including the level of NGAL in a sample and the clinical condition of the subject, the subject's body fluid can be sampled daily, or weekly, or within a few weeks, or monthly or within a few months, semi-annually, annually, or within several years, and at any interval in between. Repeat sampling can be done at a period of time after treatment to detect any change in AKI status. Sampling need not be continuous, but can be intermittent (e.g., sporadic). In some embodiments, it is not necessary to obtain and keep a sample of the bodily fluid from the subject. For example, in some embodiments, the subject can urinate onto a test strip, for example a test strip of the type used in pregnancy testing kits. In other embodiments, a sample of bodily fluid, such as blood from a pin prick, can be applied onto a test strip—for example a test strip similar to those used for blood typing.

Although generally the sample of a bodily fluid, such as blood or urine, is obtained from a subject and tested by a laboratory or by a medical professional (for example using an NGAL testing kit, e.g. a urine dipstick based kit, or an ELISA based kit), home-testing kits are also within the scope of the present invention. In one aspect, the present invention comprises a kit for performing the methods of the invention, containing, for example, a device for detecting NGAL protein in the urine, and optionally including a positive control containing NGAL protein, and optionally including instructions, for example regarding the threshold levels of NGAL above which a diagnosis of AKI can be made. The device in such kits can comprise, for example, an ELISA plate, a dipstick to be dipped in a urine sample, or a stick on which the subject should urinate. In some embodiments, such devices are configured such that they give a positive result only if the level of NGAL exceeds a threshold level, such as one of the threshold levels described herein. Methods for making and using such devices are well known in the art. Kits (ELISA kits), antibodies, and other reagents for detection of NGAL in the urine are commercially available, e.g. from Bioporto Diagnostics A/S and from R & D Systems, and can be used to make a kit according to the present invention. Such kits can be used by subjects themselves (e.g. home testing kits) or can be used by medical or laboratory staff.

The present invention also provides methods based on measuring the levels of circulating NGAL, as opposed to urinary NGAL. Blood sampling is a routine clinical procedure, and blood samples of individuals may have been stored and preserved, providing a valuable database of historical samples that may be used to predict the progression of kidney disease and/or liver disease in certain patients

According to the methods of the invention, the presence and/or amount of NGAL protein in a bodily fluid, such as urine, can be detected and/or measured using any means known in the art. For example, in one embodiment, NGAL protein can be detected using antibodies that are specific to NGAL. Any antibody, such as a monoclonal or polyclonal antibody, that binds to NGAL can be used. For example, monoclonal antibodies that bind to NGAL are described in “Characterization of two ELISAs for NGAL, a newly described lipocalin in human neutrophils”, Lars Kjeldsen et al., (1996) Journal of Immunological Methods, Vol. 198, 155-16, the contents of which are herein incorporated by reference. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002). Further non-limiting examples of antibodies that can be used to detect NGAL protein in the methods of the invention are also provided in the Examples. Antibodies that bind to NGAL are also available commercially, for example from the Antibody Shop, Copenhagen, Denmark, as HYB-211-01, HYB-211-02, and NYB-211-05. In addition, one of skill in the art can readily produce antibodies that bind to NGAL, or can have them produced by an antibody production company.

Any method can be used to detect and or measure the levels of NGAL protein, including, but not limited to, immunohistochemistry-based methods, immuno-blotting based methods, immunoprecipitation-based methods, affinity-column based methods (including immunoaffinity column based methods), ELISA-based methods, other methods in which an NGAL antibody is immobilized on a solid substrate (such as beads), and the like. In some such methods the antibody to NGAL, or a secondary or tertiary antibody that binds directly or indirectly to the NGAL antibody, can be labeled with a detectable moiety, such as a fluorescent moiety, a radioactive moiety, or a moiety that is an enzyme substrate and can be used to generate a detectable moiety, such as horse radish peroxidase. Such methods are well known in the art and can be used to detect the presence and/or measure the amount of NGAL in a bodily fluid sample, such as urine, without undue experimentation.

In circumstances where the amount of NGAL is to be measured, positive controls containing known amounts of NGAL protein can be used, for example for calibration purposes. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

In other aspects of the invention, a diagnosis of AKI or HRS, or a prediction or mortality, can be based upon, or can include, detecting the presence of NGAL protein or mRNA in the kidney itself, as opposed to in a bodily fluid such as urine, for example by detecting a high level of NGAL protein or mRNA in the kidney itself, or by detecting a specific localization of NGAL protein or mRNA within the kidney. Such methods can be used alone, or can be used in conjunction with one or more other methods, such as the methods described herein for detection of NGAL in urine or other bodily fluids or standard diagnostic methods based on the examination of kidney biopsy samples. Such methods can be performed using a kidney biopsy sample. However, methods for assessing the expression and/or localization of NGAL protein or mRNA in the kidney in situ are also provided by the invention, for example methods wherein, for example, labeled agents that bind to NGAL protein or mRNA are delivered to a subject and can be visualized in vivo, for example using imaging techniques such as CAT scan-based techniques and MRI-based techniques.

Detection of NGAL mRNA or protein in the kidney can be determined using standard techniques and methodologies known to those of skill in the art, for example using samples obtained by biopsy of the kidney. For example, NGAL mRNA can be detected by in situ hybridization using probes specific for NGAL, or by any other method known to be useful for detection of specific mRNAs, including, but not limited to, PCR-based techniques. The sequence of NGAL, including human NGAL, is known in the art. Similarly, sequences of probes and primers that can be used to detect NGAL are known in the art. In addition, NGAL protein can be detected using antibodies that are specific to NGAL, e.g. monoclonal or polyclonal antibodies can be used. In addition, detection methods that can be used, include, but are not limited to, immunohistochemistry-based methods and the like. Antibodies that are specific to NGAL and that could be used to detect NGAL in the kidneys are known in the art. Monoclonal antibodies for NGAL, are described, for example, in “Characterization of two ELISAs for NGAL, a newly described lipocalin in human neutrophils”, Lars Kjeldsen et al., (1996) Journal of Immunological Methods, Vol. 198, 155-16, herein incorporated by reference in its entirety. Non-limiting examples of antibodies that can be used to detect NGAL protein are provided in the Examples. Antibodies that bind to NGAL are also available commercially, for example from the Antibody Shop, Copenhagen, Denmark, as HYB-211-01, HYB-211-02, and NYB-211-05. Typically, HYB-211-01 and HYB-211-02 can be used with NGAL in both its reduced and unreduced forms. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002).

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

### Urinary NGAL Distinguishes Type of Kidney Failure and Predicts Mortality in Patients with Cirrhosis

### Example 1

The study described in this Example was carried out to determine whether NGAL could discriminate AKI and predict mortality in cirrhotic patients.

Patients admitted to the hospital with a diagnosis of cirrhosis were enrolled and informed consent was obtained. Urine NGAL (uNAL) was quantified by immunoblot and corrected to urine creatinine. Kidney function was determined by glomerular filtration rate (GFR) from the MDRD (Modification of Diet in Renal Disease) formula. Continuous data were log transformed for statistical testing. Comparisons were made by ANOVA and t-test for unequal variances. Test characteristics were determined by receiver operator characteristic (ROC) curve analysis.

Forty-four patients were enrolled in the study. The mean age was 59 years, and 59% of the patients were male. Cirrhosis etiologies were hepatitis C virus (HCV) (41%), alcohol (26%), HCV and alcohol (14%), cryptogenic (14%) and other (7%). uNGAL levels were significantly higher in patients with AKI in comparison to patients with PRA, CKD or normal kidney function (Table 1). During hospitalization, seven patients died, eight were admitted to the ICU, six had nephrology consultation and five required hemodialysis (HD).

TABLE 1
Markers of Kidney Function Stratified by Diagnostic Category

| | Normal | | | | | |
|---|---|---|---|---|---|---|
| | Kidney | | | PreRenal | | |
| | Function | AKI | HRS | Azotemia | CKD | F-value |
| n | 19 | 3 | 3 | 12 | 7 | |
| Serum Creatinine | | | | | | |
| mean mg/dL (SD) | 0.7 (0.2)* | 4.3 (1.9) | 2.2 (1.0) | 1.6 (0.8)* | 1.6 (0.4)* | <0.0001 |
| eGFR | | | | | | |
| mean mL/minute | 120 (33)* | 16 (8) | 29 (14) | 46 (17) | 45 (12) | <0.0001 |
| (SD) | | | | | | |
| uNGAL | | | | | | |
| mean mcg/mg | 27 (21)* | 1173 (1183) | 178 (251) | 26 (18)* | 33 (49)† | <0.0001 |
| creatinine (SD) | | | | | | |

In comparison to AKI:
*p < 0.05,
† p < 0.01

Mean uNGAL was significantly higher in patients who died in comparison to patients who did not die (549 (860) μg/gm v. 28 (27) mg/dL, p<0.02 respectively). Analysis of the area under the ROC curve (AUC) for mortality was 0.847 and 0.817 for uNGAL and Scr respectively. At a cutoff of 130 μg/gm for uNGAL and 2.2 mg/dL for SCr, sensitivity and specificity were 57% and 98% respectively to discriminate inpatient mortality. Logistic regression demonstrated that a 10 μg/gm rise in uNGAL increased the odds of mortality by 17% (Odds Ratio (OR) 1.17, 95% Confidence Interval (CI) 1.00-1.37). The odds of death were 48-fold higher for patients with a uNGAL>130 μg/gm (OR 48.00, 95% CI 3.99-577.64).

In the ongoing study in patients with cirrhosis described in this Example, uNGAL measurement may be used to discriminate AKI from other forms of kidney dysfunction and can predict in-patient mortality.

### Example 2

Kidney failure is an important predictor of mortality among patients with cirrhosis. Serum creatinine (scr) poorly distinguishes acute kidney injury (AKI) from hepatorenal syndrome (HRS), prerenal azotemia (PRA) or stable chronic kidney disease (CKD), especially in the setting of the edema that is characteristic of portal hypertension. Empiric hydration may be dangerous in these patients as it may worsen portal hypertension. Urine neutrophil gelatinase associated lipocalin (uNGAL) is highly sensitive and specific for the diagnosis of AKI in non-cirrhotic patients. Prior to the present invention, the characteristics of uNGAL in the setting of cirrhosis or HRS was not known.

Consecutive hospitalized cirrhotic patients were enrolled and informed consent was obtained. Patients with end-stage real disease (ESRD) were excluded. Urine was collected within 24 hrs of admission and patient data was collected for the duration of their inpatient stay. uNGAL was measured by immunoblot and corrected to urine creatinine. Kidney function was determined by MDRD glomerular filtration rate (GFR). The category of kidney function (e.g. AKI, PRA, HRS, CKD or normal) was determined by a nephrologist blinded to uNGAL levels. Continuous data were log transformed prior to statistical testing. Comparisons were made by ANOVA and t-test for unequal variances. Test characteristics were determined by receiver operator characteristic (ROC) curve analysis.

Table 2 below summarizes characteristics of the patients in the study.

TABLE 2
Patient Characteristics

| | |
|---|---|
| Male (%) | 26 (59%) |
| Age, mean ± SD | 58.7 ± 9.7 |
| Etiology of Cirrhosis (%): | |
| Hepatitis C (HCV) | 18 (41%) |
| Alcohol | 11 (26%) |
| HCV and alcohol | 6 (14%) |
| Cryptogenic | 6 (14%) |
| Other | 3 (7%) |

(N = 44)

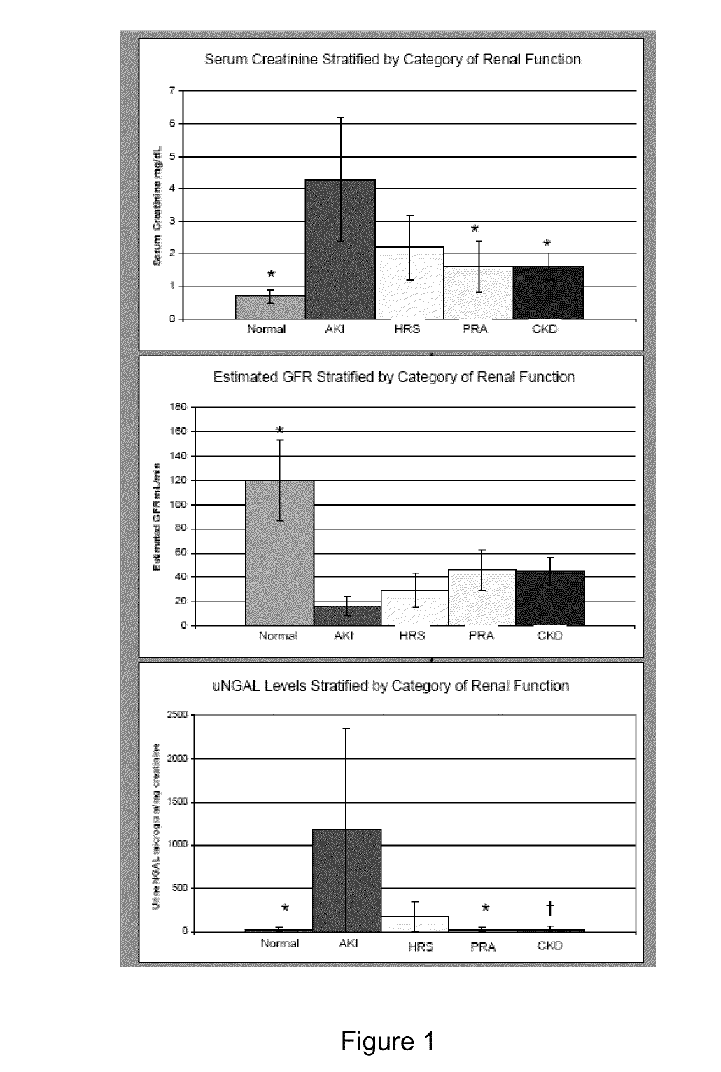

FIG. 1 shows some of the results in graphical form. Serum Creatinine (mg/dL), eGFR (mL/min), and uNGAL levels (microgram/g creatinine) are shown, stratified by category of renal function. In comparison to AKI: *p<0.05, † p<0.01. Patients with AKI had significantly greater serum creatinine (scr) compared to patients with PRA, CKD or normal kidney function. uNGAL was significantly elevated when compared to other categories of kidney function.

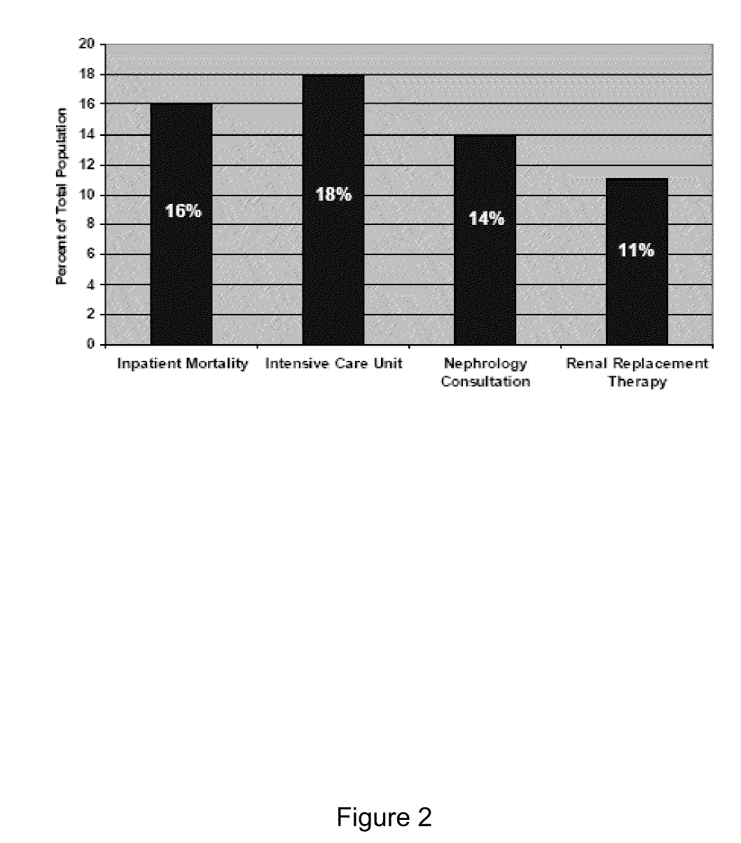

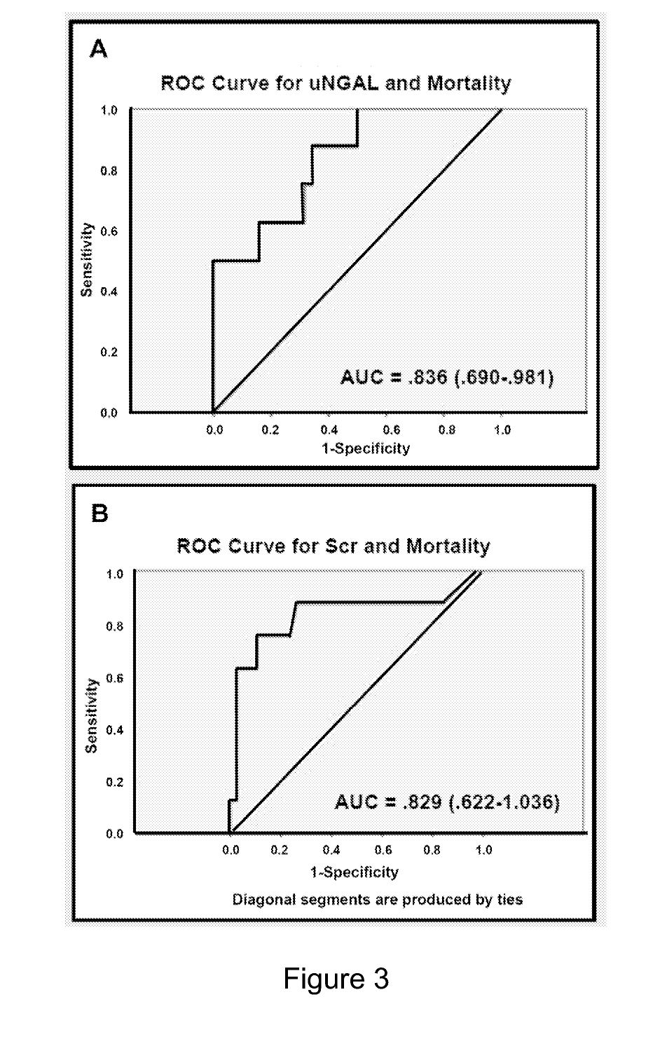

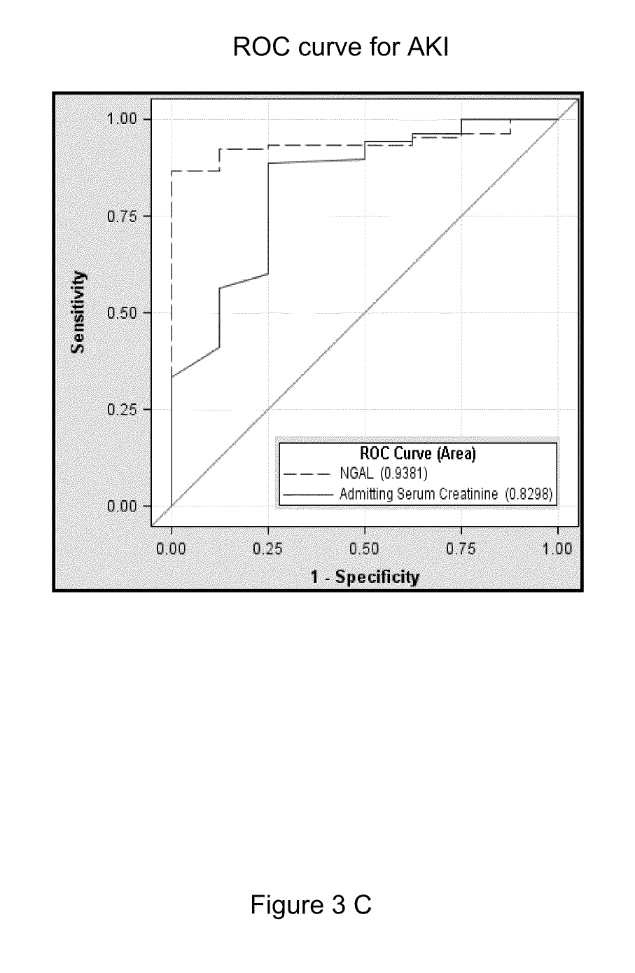

FIG. 2 shows the clinical outcomes of all enrolled patients. FIG. 3 shows the ROC analysis for uNGAL and Scr in predicting mortality and AKI.

Table 3 below provides data showing that uNGAL measurements predict mortality.

TABLE 3
uNGAL to Predict Mortality

| Mean (SD) uNGAL (μg/gm): | |
|---|---|
| Expired during admission | 549 (860)* |
| Survived to discharge | 28 (27) |
| For uNGAL cutoff of 130 micrograms/gram or scr 2.2: | |
| Sensitivity | 57% |
| Specificity | 98% |
| For uNGAL cutoff of 130 μg/gm: | |
| OR of death (95% CI) | 48 (4-578) |
| For every 10 μg/gm rise in uNGAL: | |
| OR of death (95% CI) | 1.17 (1-1.37) |

*uNGAL was significantly different between those patients who expired during admission and those who survived (*p < 0.02).

In patients with cirrhosis, uNGAL measurements discriminated AKI from other forms of kidney dysfunction and predicted inpatient mortality. uNGAL can also help identify patients with HRS. uNGAL can be helpful in the early assessment and treatment of patients with cirrhosis and renal failure, and guide targeted treatment of renal failure in these patients.

### Example 3

In patients with cirrhosis, kidney failure is among the strongest predictors of mortality and creatinine is one of three variables in the MELD score used for liver allocation. Determining etiology of kidney failure in patients with cirrhosis and edema is a clinical challenge. Urine NGAL (uNGAL) predicts the onset and severity of acute kidney injury (AKI) as well as mortality in many clinical settings. However, prior to the present invention, it was not known if uNGAL could be used to discriminate between types of kidney failure in patients with cirrhosis.

A prospective study of adults admitted to either general medicine or medical intensive care units (ICUs) with cirrhosis (biopsy proven or combination of imaging, laboratory and clinical evidence with documentation of cirrhosis on 2 distinct clinical notes) was performed.

Patients with end-stage renal disease (ESRD), and/or who were anuric, had a proteinuria >500 mg daily, a urinary tract infection, HIV, or a previous transplant, were excluded from the study. After informed consent, 1 ml of urine was collected within 24 hours of admission and frozen.

Primary outcomes assessed were AKI, HRS and PRA. Secondary outcomes assessed were inpatient dialysis (HD), ICU admission, and nephrology consultation.

Urine was processed and uNGAL measured using a commercially available ELISA-based assay (Antibodyshop, Denmark). uNGAL measurements were corrected for urine creatinine.

Statistical tests/methods used include ANOVA and Tukey tests, and linear and logistic regression. Patients with indeterminate renal function (n=7) were excluded from the final analysis. GFR was estimated using the MDRD equation.

The Tables below show some further results generated in the study.

TABLE 4
Patient Characteristics

| | |
|---|---|
| Age, mean (SD) | 56.2 (11) |
| Male gender (%) | 70 (61) |
| Etiology of Cirrhosis (%): | |
| Hepatitis C (HCV) | 49 (43) |
| Alcohol | 24 (21) |
| HCV and alcohol | 13 (12) |
| Cryptogenic/NAFLD | 12 (11) |
| HBV | 5 (4) |
| Autoimmune hepatitis | 5 (4) |
| Other | 7 (5) |
| Ethnicity: | |
| Non-Hispanic White | 33 (30) |
| Hispanic White | 52 (48) |
| African American | 14 (13) |
| Other | 10 (9) |
| Complications of cirrhosis (%): | |
| Ascites | 72 (65) |
| Varices | 59 (54) |
| Hepatocellular carcinoma | 20 (18) |
| TIPS | 15 (14) |
| MELD on admission, median (IQR) | 17 (11) |
| History of CKD | 19 (18) |
| Length of hospitalization (days), median (IQR) | 6 (10) |

(N = 115)

TABLE 5

| | | | uNGAL | | |
|---|---|---|---|---|---|
| Renal | | Mortality | (mcg/g | Creatinine | GFR |
| category | Frequency (%) | (%) | cr) | (mg/dl) | (ml.min) |
| Normal | 49 (43) | 1/49 | 58 (96) | 0.8 (0.2) | 106 (27) |
| | | (2%) | | | |
| Stable | 13 (11) | 1/13 | 111 (192) | 1.5 (0.4) | 47 (14) |
| CKD | | (8%) | | | |
| PRA | 30 (26) | 1/30 | 67 (155) | 1.7 (0.8) | 45 (17) |
| | | (3%) | | | |
| HRS | 13 (11) | 7/13 | 318 (349)*** | 2.2 (1.1) | 37 (21) |
| (I/II) | | (54%) | | | |
| AKI | 10 (9) | 4/10 | 1069 (1153)*** | 3.0 (1.9)** | 33 (25) |
| | | (40%) | | | |

TABLE 6
ln(uNGAL) to Predict Outcomes in Univariate Analysis

| Outcome | OR | 95% CI | P value |
|---|---|---|---|
| Inpatient morality | 1.90 | 1.30-2.76 | 0.0009 |
| Dialysis | 1.61 | 1.09-2.40 | 0.02 |
| Mortality or | 1.89 | 1.32-2.69 | 0.004 |
| dialysis | | | |
| Intensive care unit | 1.43 | 1.07-1.91 | 0.01 |
| Renal Consultation | 1.84 | 1.31-2.58 | 0.004 |

TABLE 7
Multiple Logistic Regression to Predict Inpatient Mortality

| Predictor | OR | 95% CI | P value |
|---|---|---|---|
| uNGAL ≧165 | 5.01 | 1.30-19.40 | 0.02 |
| mcg/g cr | | | |
| Age (years) | 1.00 | 0.93-1.07 | 1.0 |
| HRS renal group | 8.94 | 2.07-38.70 | 0.003 |
| Creatinine ≧1.2 | 4.41 | 0.77-25.12 | 0.09 |
| mg/dl | | | |

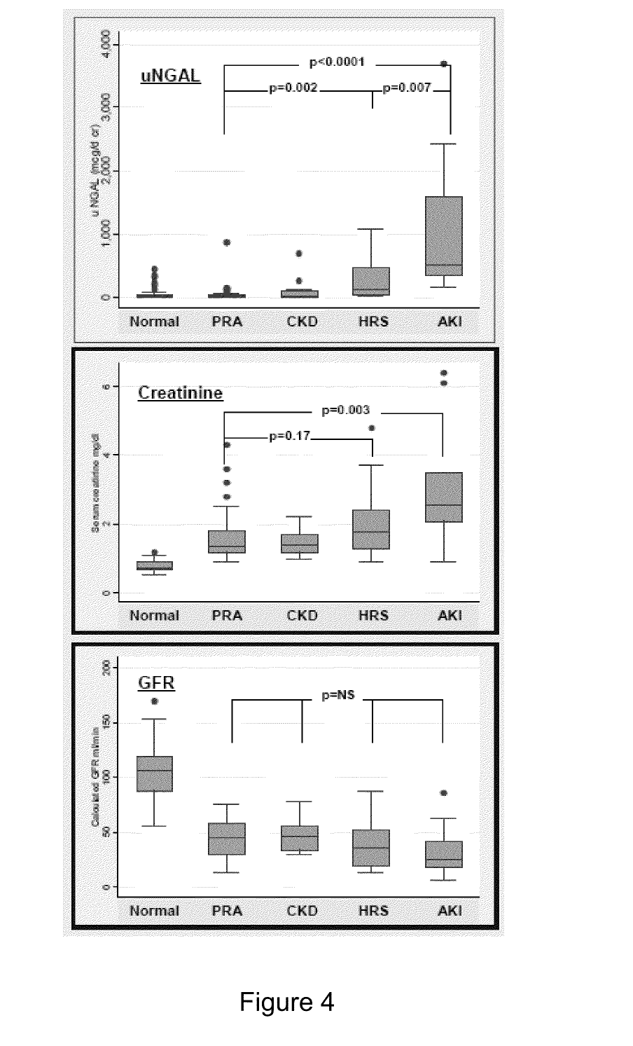

FIG. 4 illustrates some of the data from the study in graphical form.

In this study of patients with cirrhosis, a single uNGAL measurement on admission distinguished between AKI, HRS and PRA, and predicted renal related inpatient outcomes. uNGAL is an independent predictor of inpatient mortality even after adjustment for other strong risk factors of mortality. Serum creatinine did not predict mortality after adjustment for other risk factors associated with mortality. HRS is strongly associated with mortality

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

What is claimed is:

1. A method for determining whether a subject with cirrhosis has acute kidney injury (AKI) or a non-AKI kidney condition, the method comprising determining the concentration of NGAL protein in a urine sample from a subject with cirrhosis, wherein a concentration of NGAL in the urine sample that exceeds a threshold amount indicates that the subject has AKI, and wherein a concentration of NGAL in the urine sample that is less than the threshold amount indicates that the subject has a non-AKI kidney condition or has normal kidney function.

2. The method of claim 1, wherein the threshold amount is between about 500 and about 1000 micrograms per gram creatinine.

3. The method of claim 1, wherein the determining step comprises performing immunoassay to detect NGAL protein.

4. The method of claim 1, wherein the non-AKI kidney condition is hepatorenal syndrome (HRS), prenal azotemia (PRA) or chronic kidney disease (CKD).

5. The method of claim 1, further comprising adjusting the subject's treatment regimen based on whether the concentration of NGAL in the urine sample exceeds or is less than the threshold amount.

6. A method for determining whether a subject with cirrhosis has hepatorenal syndrome (HRS) or a non-HRS kidney condition, the method comprising determining the concentration of NGAL protein in a urine sample from a subject with cirrhosis, wherein a concentration of NGAL in the urine sample that falls within a certain range indicates that the subject has HRS, and wherein a concentration of NGAL in the urine sample that is greater or less than the range indicates the subject has a non-HRS kidney condition or has normal kidney function.

7. The method of claim 6, wherein the range is from about 150 to about 500 micrograms per gram creatinine.

8. The method of claim 6, wherein a concentration of NGAL in the urine sample that is greater than the range indicates the subject has AKI.

9. The method of claim 6, wherein a concentration of NGAL in the urine sample that is less than the range indicates the subject has prenal azotemia (PRA), chronic kidney disease (CKD), or normal kidney function.

10. The method of claim 6, wherein the determining step comprises performing immunoassay to detect NGAL protein.

11. The method of claim 6, further comprising adjusting the subject's treatment regimen based on whether the concentration of NGAL in the urine sample exceeds or is less than the threshold amount.

12. A method for predicting disease-specific mortality in a subject with cirrhosis, the method comprising determining the amount of NGAL protein in a urine sample from a subject with cirrhosis, wherein an amount of NGAL protein in the urine sample that exceeds a threshold amount indicates the subject has a high chance of disease-specific mortality, and an amount of NGAL protein in the urine sample that is less than the threshold amount indicates that the subject has a low chance of disease-specific mortality.

13. The method of claim 12, wherein the threshold amount is between about 10 and about 180 micrograms per gram creatinine.

14. The method of claim 12, wherein the threshold amount is about 130 micrograms per gram creatinine.

15. The method of claim 12, wherein the urine sample is obtained during an acute episode of liver and/or kidney disease, and wherein an amount of NGAL protein in the urine sample that exceeds the threshold amount indicates the subject has a high chance of disease-specific mortality during the acute episode, and wherein an amount of NGAL protein in the urine sample that is less than the threshold amount indicates that the subject has a low chance of disease-specific mortality during the acute episode.

16. The method of claim 12, wherein the urine sample is obtained during a hospitalization for liver and/or kidney disease, wherein an amount of NGAL protein in the urine sample that exceeds the threshold amount indicates the subject has a high chance of disease-specific mortality during the hospitalization, and wherein an amount of NGAL protein in the urine sample that is less than the threshold amount indicates that the subject has a low chance of disease-specific mortality during the hospitalization.

17. The method of claim 12, wherein a subject with an amount of NGAL protein in the urine sample that exceeds the threshold amount has a 20- to 80-fold higher chance of disease-specific mortality than a subject having an amount of NGAL protein in the urine sample that is less than the threshold amount.

18. The method of claim 12, wherein the determining step comprises performing immunoassay to detect NGAL protein.

19. The method of claim 12, further comprising adjusting the subject's treatment regimen based whether the concentration of NGAL in the urine sample exceeds or is less than the threshold amount.

20. A method for determining whether a subject with cirrhosis has AKI, HRS, or PRA, the method comprising:
(a) determining the amount of NGAL protein in the urine of a subject with cirrhosis, and
(b) determining the subject's fractional excretion of sodium (FENa), wherein subjects having a uNGAL level lower than about 60 mg/mL are likely to have PRA,
and wherein subjects having a uNGAL level higher than about 60 mg/mL and a FENa value of less than about 0.5% are likely to have HRS,
and wherein subjects having a uNGAL level higher than about 60 mg/mL and a FENa value of greater than about 0.5% are likely to have AKI.
